(19) United States
(12) Patent Application Publication
(10) Pub. No.: US 2003/0087257 A1
(43) Pub. Date: May 8, 2003

(54) Method for assembling of fragments in DNA sequencing

(76) Inventors: Pavel A. Pevzner, La Jolla, CA (US); Haixu Tang, Los Angeles, CA (US); Michael S. Waterman, Dayton, NV (US)

Correspondence Address: Rajiv, Yadav, Ph.D., Esq., McCutchen, Doyle, Brown & Enersen, LLP, 18th Floor, Three Embarcadero Center, San Francisco, CA, 94111, US

(21) Appl. No.: 10/120,931

(22) Filed: Apr. 10, 2002

Related U.S. Application Data
(60) Provisional application No. 60/285,059, filed on Apr. 19, 2001.

(51) Int. Cl. C12Q 1/68; G06F 19/00; G01N 33/48; G01N 33/50

(52) U.S. Cl. 435/6; 702/20

(57) ABSTRACT

A new Eulerian Superpath approach to fragment assembly in DNA sequencing that resolves the problem of repeats in fragment assembly is disclosed. The invention provides for the reduction of the fragment assembly to a variation of the classical Eulerian path problem. This reduction opens new possibilities for repeat resolution and allows one to generate provably optimal error-free solutions of the large-scale fragment assembly problems.

### CROSS REFERENCED TO RELATED APPLICATION

This application claims the benefit of provisional application Ser. No. 60/285,059 filed Apr. 19, 2001, the disclosure of which is incorporated by reference in its entirety.

### FIELD OF THE INVENTION

This invention pertains to the field of bioinformatics. More particularly, it describes a method for assembling fragments in sequencing of a deoxyribonucleic acid (DNA).

### BACKGROUND OF THE INVENTION

For the last twenty years fragment assembly in DNA sequencing mainly followed the “overlap-layout-consensus” paradigm used in all currently available software tools for fragment assembly. See, e.g., P. Green, Documentation for Phrap. http://www.genome.washington.edu/UWGC/analysis-tools/phrap.htm, (1994); J. K. Bonfield, K. F. Smith and R. Staden, A New DNA Sequence Assembly Program, Nucleic Acids Research, vol. 23, pp. 4992-4999 (1995); G. Sutton, et. al., TIGR Assembler: A New Tool for Assembling Large Shotgun Sequencing Projects, Genome Science & Technology, vol. 1, pp 9-19 (1995); X. Huang and A. Madan, CAP-3: A DNA Sequence Assembly Program, Genome Research, vol. 9, pp. 868-877 (1999).

Although this approach proved to be useful in assembling contigs of moderate sizes, it faces difficulties while assembling prokaryotic genomes a few million bases long. These difficulties led to introduction of the double-barreled DNA sequencing. See, e.g., J. Roach, et. al., Pairwise End Sequencing: A Unified Approach to Genomic Mapping and Sequencing, Genomics, vol. 26, pp. 345-353 (1995); J. Weber and G. Myers, Whole Genome Shotgun Sequencing, Genome Research, vol. 7, pp. 401-409 (1997). The double-barreled DNA sequencing uses additional experimental information for assembling large genomes in the framework of the same “overlap-layout-consensus” paradigm. See, e.g., E. W. Myers, et. al., A Whole Genome Assembly of Drosophila, Science, vol. 287, pp. 2196-2204 (2000).

Although the classical approach culminated in some excellent fragment assembly tools (Phrap, CAP3, TIGR, and Celera assemblers being among them), critical analysis of the “overlap-layout-consensus” paradigm reveals some weak points. First, the overlap stage finds pair-wise similarities that do not always provide true information on whether the fragments (sequencing reads) overlap. A better approach would be to reveal multiple similarities between fragments since sequencing errors tend to occur at random positions while the differences between repeats are always at the same positions. However, this approach is not feasible due to high computational complexity of the multiple alignment problem. Another problem with the conventional approach to fragment assembly is that finding the correct path in the overlap graph with many false edges (layout problem) becomes very difficult.

Clearly, these problems are difficult to overcome in the framework of the “overlap-layout-consensus” approach and the existing fragment assembly algorithms are often unable to resolve the repeats even in prokaryotic genomes. Inability to resolve repeats and to figure out the order of contigs leads to additional experimental work to complete the assembly. See, H. Tettelin, Optimized Multiplex PCR: Efficiently Closing a Whole-Genome shotgun sequencing project, Genomics, vol. 62, pp. 500-507 (1999). Moreover, all the tested programs made errors while assembling shotgun reads from the bacterial sequencing projects for Campylobacter jejuni, see, J. Parkhill, et. al., The Genome Sequence of the Food-Borne Pathogen Campylobacter jejuni Reveals Hypervariable Sequences, Nature, vol. 403, pp. 665-668 (2000); for Neisseria meningitidis (NM), see, J. Parkhill, et. al., Complete DNA Sequence of a Serogroup a Strain of Neisseria meningitidis Z2491, Nature, vol. 402, pp. 502-506 (2000); and for Lactococcus lactis, see, A. Bolotin, et. al., Low-Redundancy Sequencing of the Entire Lactococcus lactis IL1403 Genome, Antonie van Leeuwenhoek, vol. 76, pp. 27-76 (1999).

Those with reasonable skills in the art are well aware of potential assembly errors and are forced to carry additional experimental tests to verify the assembled contigs. They are also aware of assembly errors as evidenced by finishing software that supports experiments correcting these errors. See, D. Gordon, C. Abajian, and P. Green, Consed: A Graphical Tool for Sequence Finishing. Genome Research, vol. 8(3) , pp. 195-202 (1998).

Another area of DNA arrays provides assistance in resolving these problems. Sequencing by Hybridization (SBH) is a method helpful in this respect. Conceptually, SBH is similar to fragment assembly, the only difference is that the “reads” in SBH are much shorter l-tuples (contiguous sting of letters/nucleotides of length l). In fact, the very first attempts to solve the SBH fragment assembly problem followed the “overlap-layout-consensus” paradigm. See, R. Drmanac, et. al., Sequencing of Megabase Plus DNA by Hybridization: Theory of the Method, Genomics, vol. 4, pp. 114-128 (1989); Y. Lysov, et. al., DNA Sequencing by Hybridization with oligonucleotides, Doklady Academy Nauk USSR, vol. 303, pp. 1508-1511, (1988). However, even in a simple case of error-free SBH data, this approach faces serious difficulties and the corresponding layout problem leads to the NP-complete Hamiltonian Path Problem. A different approach was proposed that reduces SBH to an easy-to-solve Eulerian Path Problem in the de Bruijn graph by abandoning the “overlap-layout-consensus” paradigm. See, P. Pevzner, L-Tuple DNA Sequencing: Computer Analysis, Journal of Biomolecular Structure and Dynamics, vol. 7, pp. 63-73 (1989).

Since the Eulerian path approach transforms a once difficult layout problem into a simple one, attempts were made to apply the Eulerian path approach to fragment assembly. For instance, the fragment assembly problem was mimicked as an SBH problem, where every read of length n was represented as a collection of (n−l+1) l-mers and an Eulerian path algorithm was applied to a set of l-tuples formed by the union of such collections for all reads. See, R. Idury and M. Waterman, A New Algorithm for DNA Sequence Assembly, Journal of Computational Biology, vol. 2, pp. 291-306 (1995). At the first glance this transformation of every read into a collection of l-tuples is a very shortsighted procedure since information about the sequencing reads is lost. However, the loss of information is minimal for large l and is well paid for by the computational advantages of the Eulerian path approach in the resulting easy-to-analyze graph. In addition, the lost information can be easily restored at the later stages.

However, the Idury-Waterman approach, while very promising, did not scale up well. The problem is that the sequencing errors transform a simple de Bruijn graph (corresponding to an error-free SBH) into a tangle of erroneous edges. For a typical sequencing project, the number of erroneous edges is a few times larger than the number of real edges and finding the correct path in this graph is extremely difficult, if not impossible, task. Moreover, repeats in prokaryotic genomes pose serious challenges even in the case of error-free data since the de Bruijn graph gets very tangled and difficult to analyze.

In view of the above-described drawbacks and disadvantages of the classical “overlap-layout-consensus” approach, a better method for fragment assembly in DNA sequences is needed. There is no known method taught by prior art allowing fragment assembly while free of the problems discussed above. Yet, a need for such better method is acute.

This invention teaches such new method based on a new Eulerian superpath approach. The main result is the reduction of the fragment assembly problem to a variation of the classical Eulerian path problem. This reduction opens new possibility for repeat resolution and leads to the EULER software that generated probably optimal solutions for the large-scale assembly projects that were studied.

### SUMMARY OF THE INVENTION

This invention teaches how to decide whether two similar reads correspond to the same region, that is, whether the differences between the two reads are due to sequencing errors or to two copies of a repeat located in different parts of the genome. Solving this problem is important for all fragment assembly algorithms, as pair-wise comparison used in conventional algorithms does not adequately resolve this problem.

An error-correction procedure is described which implicitly uses multiple comparison of reads and successfully distinguishes these two situations.

There were attempts in prior art to deal with errors and repeats via graph reductions. See, R. Idury and M. Waterman, A New Algorithm for DNA Sequence Assembly, Journal of Computational Biology, vol. 2, pp. 291-306; and E. M. Myers, Toward Simplifying and Accurately Formulating Fragment Assembly, Journal of Computational Biology, vol. 2, pp. 275-290 (1990). However, both these methods do not explore multiple alignment of reads to fix sequencing errors at the pre-processing stage. Of course, multiple alignment of reads is costly and pair-wise alignment is the only realistic option at the overlap stage of the conventional fragment assembly algorithms. However, the multiple alignment becomes feasible when one deals with perfect or nearly perfect matches of short l-tuples, exactly the case in the SBH approach to fragment assembly.

This invention describes an error correction method utilizing the multiple alignment of short substrings to modify the original reads and to create a new instance of the fragment assembly problem with the greatly reduced number of errors. This error correction approach makes the reads almost error-free and transforms the original very large graph into a de Bruijn graph with very few erroneous edges. In some sense, the error correction is a variation of the consensus step taken at the very first step of fragment assembly (rather than at the last one as in the conventional approach).

Even in an ideal situation, when the error-correction procedure eliminated all errors, and one deals with a collection of error-free reads, there exists no algorithm to reliably assemble such error-free reads in a large-scale sequencing project. For example, Phrap, CAP3 and TIGR assemblers make 17, 14, and 9 assembly errors, respectively, while assembling real reads from the N. meningitidis genome. All algorithms known in prior art still make errors while assembling the error-free reads from the N. meningitidis genome. Although the TIGR assembler makes less errors than other programs, this accuracy does not come for free, since this program, produces twice as many contigs as do the other programs.

In comparison, EULER made no assembly errors and produced less contigs with real data than other programs produced with error-free data. Moreover, EULER allows one to reduce the coverage by 30% and still produce better assemblies than other programs with full coverage. EULER can be also used to immediately improve the accuracy of Phrap, CAP3 and TIGR assemblers: these programs produce better assemblies if they use error-corrected reads from EULER.

To achieve such accuracy, EULER has to overcome the bottleneck of the Idury-Waterman approach mentioned above and to restore information about sequencing reads that was lost in the construction of the de Bruijn graph.

The second aspect of this invention, Eulerian Superpath approach, addresses the above identified problem. Every sequencing read corresponds to a path in the de Bruijn graph called a read-path. An attempt to take into account the information about the sequencing reads leads to the problem of finding an Eulerian path that is consistent with all read-paths, an Eulerian Superpath Problem. A method to solve this problem is discussed subsequently

According to one aspect of this invention, a method is offered for correcting errors in original sequencing reads of a DNA, the method comprising a step of performing an error detection and correction, the error correction being conducted prior to assembling such reads in a contiguous piece.

According to another aspect of this invention, a method of assembling a puzzle from original pieces thereof is offered, the method comprising a step of altering said original pieces.

According to yet another aspect of this invention, in the above mentioned method of assembling a puzzle from original pieces, the puzzle comprises a DNA sequence.

According to yet another aspect of this invention, in the above mentioned methods for correcting errors in original sequencing reads of a DNA and of assembling a puzzle from original pieces, the step of altering comprises dismembering the original pieces into sub-pieces, each sub-piece being smaller in size than the original piece from which said sub-piece originated.

According to yet another aspect of this invention, in the above mentioned method of assembling a puzzle from original pieces where the puzzle comprises a DNA sequence, the original pieces comprise reads of the DNA sequence.

### BRIEF DESCRIPTION OF THE DRAWINGS

FIG. 1 is a schematic diagram showing how an error in a read affects l-tuples in the read and l-tuples in the complementary read.

FIG. 2 is a schematic diagram showing a repeat and a system of paths overlapping with the repeat.

FIG. 3 is a schematic diagram showing an x,y-detachment as being an equivalent transformation reducing the number of edges in the graph.

FIG. 4 is a schematic diagram showing an x,y1-detachment where x is a multiple edge.

FIG. 5 is a schematic diagram showing some particular examples of graphs.

FIG. 6 is a schematic diagram showing some other particular examples of graphs.

FIG. 7 is a schematic diagram showing a detachment where x is a removable edge.

FIG. 8 demonstrates comparative analysis of EULER, Phrap, CAP and TIGR assemblers as well as EULER on real and simulated low-coverage sequencing data for N. meningitidis.

### DETAILED DESCRIPTION OF THE INVENTION

Error Correction

Sequencing errors make implementation of the SBH-style approach to fragment assembly difficult. To bypass this problem, by solving the Error Correction Problem, the error rate is reduced by a factor of 35-50 at the pre-processing stage and the data are made almost error-free. As an example, the N. meningitidis (NM) sequencing project completed at the Sanger Center and described in the above mentioned prior art reference, J. Parkhill, et. al., Complete DNA Sequence of a Serogroup a Strain of Neisseria meningitidis Z2491, Nature, vol. 402, pp. 502-506, is used.

NM is one of the most “difficult-to-assemble” bacterial genome completed so far. It has 126 long perfect repeats up to 3832 bp in length, and many imperfect repeats. The length of the genome is 2,184,406 bp. The above mentioned sequencing project resulted in 53,263 reads of average length 400 (average coverage is 9.7). There were 255,631 errors overall distributed over these reads. It results in 4.8 errors per read (error rate of 1.2).

Let s be a sequencing read (with errors) derived from a genome G. If the sequence of G is known, then the error correction in s can be done by aligning the read s against the genome G. In real life, the sequence of G is not known until the very last “consensus” stage of the fragment assembly. To assemble a genome it is highly desirable to correct errors in reads first, but to correct errors in reads one has to assemble the genome first.

To bypass this catch-22, let's assume that, although the sequence of G is unknown, the set Gₗ of all continuous strings of fixed length l (l-tuples) present in G is known. Of course, Gₗ is unknown either, but Gₗ can be reliably approximated without knowing the sequence of G. An l-tuple is called solid if it belongs to more than M reads (where M is a threshold) and weak otherwise. A natural approximation for Gₗ is the set of all solid l-tuples from a sequencing project.

Let T be a collection of l-tuples called a spectrum. A string s is called a T-string if all its l-tuples belong to T. The method of error correction of this invention leads to the following.

Spectral Alignment Problem.

Given a string s and a spectrum T, find the minimum number of mutations in s that transform s into a T-string.

In the context of error corrections, the solution of the Spectral Alignment Problem makes sense only if the number of mutations is small. In this case the Spectral Alignment Problem can be efficiently solved by dynamic programming even for large l. This was not a case when similar problem was recently considered in a different context of resequencing by hybridization. See, I. Pe'er and R. Shamir, Spectrum Alignment: Efficient Resequencing by Hybridization, Proceedings of the Eighth International Conference on Intelligent Systems for Molecular Biology, pp. 260-268, San Diego (2000).

Spectral alignment of a read against the set of all solid l-tuples from a sequencing project, suggests the error corrections that may change the sets of weak and solid l-tuples. Iterative spectral alignments with the set of all reads and all solid l-tuples gradually reduce the number of weak l-tuples, increase the number l-tuples of solid l-tuples, and lead to elimination of up to about 97% of many errors in bacterial sequencing projects.

Although the Spectral Alignment Problem helps to eliminate errors (and is used as one of the steps in EULER, as subsequently discussed), it does not adequately capture the specifics of the fragment assembly. The Error Correction Problem described below is somewhat less natural than the Spectrum Alignment Problem but it is probably a better model for fragment assembly (although it is not a perfect model either).

Given a collection of reads (strings) S={(s₁, . . . Sₙ} from a sequencing project and an integer l, the spectrum of S is a set Sₗ of all l-tuples from the reads sₗ, . . . , sₙ and \check{S}ₗ, . . . \check{S}ₙ where \check{S} denotes a reverse complement of read s. Let Δ be an upper bound on the number of errors in each DNA read. A more adequate approach to error correction motivates the following

Error Correction Problem.

Given S, Δ, and l, introduce up to Δ corrections in each read in S in such a way that |Sₗ| is minimized.

An error in a read s affects at most l l-tuples in s and l l-tuples in \check{S} because such error in a read affects l l-tuples in this read and l-tuples in the complementary read, creating 2l erroneous l-tuples.

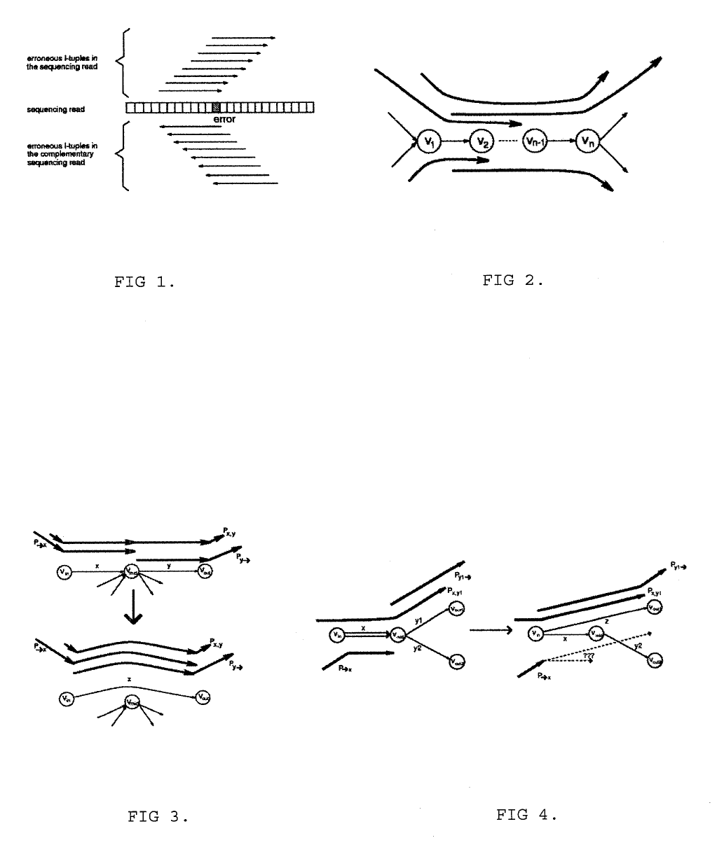

As shown on FIG. 1, such error usually creates 2l erroneous l-tuples that point out to the same sequencing error (2d for positions within a distance d<l from the endpoint of the reads). Therefore, a greedy approach for the Error Correction Problem is to look for an error correction in the read s that reduces the size of Sₗ by 2l (or 2d for positions close to the endpoints of the reads). This simple procedure already eliminates about 86.5% of errors in sequencing reads.

Below is described a more involved approach that eliminates about 97.7% of sequencing errors. This approach transforms the original fragment assembly problem with 4.8 errors per read on average into an almost error-free problem with 0.11 errors per read on average.

Two l-tuples are called neighbors if they are one mutation apart. For an l-tuple a define its multiplicity m(a) as the number of reads in S containing this l-tuple. An l-tuple is called an orphan if (i) it has small multiplicity, i.e., m(a) ≦M, where M is a threshold, (ii) it has the only neighbor b, and (iii) m(b) ≧m(a). The position where an orphan and its neighbor differ is called an orphan position. A sequencing read is orphan-free if it contains no orphans.

An important observation is that each erroneous l-tuple created by a sequencing error usually does not appear in other reads and is usually one mutation apart from a real l-tuple (for an appropriately chosen l). Therefore, a mutation in a read usually creates 2l orphans. This observation leads to an approach that corrects errors in orphan positions within the sequencing reads, if (i) such corrections reduce the size of Sₗ by more than (2l-δ), where δ is a fixed threshold, and (ii) the overall number of error corrections in a given read to make it orphan-free is at most Δ. The greedy orphan elimination approach to the Error Correction Problem starts error corrections from the orphan positions that reduce the size of Sₗ by 2l (or 2d for positions at distance d<l from the end points of the reads). After correcting all such errors the “2l condition” gradually transforms into a weaker 2l-δ condition.

Error Correction and Data Corruption.

The error-correction procedure of this invention is not perfect while deciding which nucleotide, among, for instance, A or T is correct in a given l-tuple within a read. If the correct nucleotide is A, but T is also present in some reads covering the same region, the error-correction procedure may assign T instead of A to all reads, i.e., to introduce an error, rather than to correct it, particularly, in the low-coverage regions.

The method of this invention may at times introduce errors, which is acceptable as long as the errors from overlapping reads covering the same position are consistent (i.e., they correspond to a single mutation in a genome). It is much more important to make sure that a competition between A and T is eliminated at this stage, thus reducing the complexity of the de Bruijn graph. In this way false edges are eliminated in the graph and the correct nucleotide can be easily reconstructed at the final consensus stage of the algorithm.

For N. meningitidis sequencing project, orphan elimination corrects 234,410 errors, and introduces 1,452 errors. It leads to a tenfold reduction in the number of sequencing errors (0.44 errors per read). The orphan elimination procedure is usually run with M=2 since orphan elimination with M=1 leaves some errors uncorrected. For a sequencing project with coverage 10 and error rate 1%, every solid 20-tuple has on average 2 orphans, o₁ and o₂, each with multiplicity 1 (i.e., an expected multiplicity of this 20-tuple is 8 rather than 10 as in the case of error-free reads). With some probability, the same errors in (different) reads correspond to the same position in the genome thus “merging o₁ and o₂ into a single l-tuple o with m(o)=2. Although the probability of such event is relatively small, the overall number of such cases is large for large genomes. In studies of bacterial genomes setting M =2 and simultaneous correction of up to M multiple errors worked well in practice. With M=2, additional 705 errors have been eliminated and 131 errors have been created (21,837 errors, or 0.41 errors per read are left).

Orphan elimination is a more conservative procedure than spectral alignment. Orphans are defined as l-tuples of low multiplicity that have only one neighbor. The latter condition (that is not captured by the spectral alignment) is important since in the case of multiple neighbors it is not clear how to correct an error in an orphan. For the N. meningitidis genome there were 1,862 weak 20-mers with low multiplicities (M≦2) that had multiple neighbors. The approach of this invention to this problem is to increase l in a hope that there is only one “competing” neighbor for longer l. After increasing l from 20 to 100, the number of orphans with multiple neighbors has been reduced from 1,862 to 17. Orphan elimination should be done with caution since errors in reads are sometimes hard to distinguish from differences in repeats. If the differences between repeats (particularly repeats with low coverage) are treated as errors, then orphan elimination would correct the differences between repeats instead of correcting errors. This may lead to inability to resolve repeats at the later stages. It is important to realize that error corrections in orphan positions often create new orphans. For example, a read containing an imperfect low-coverage (≦M) copy of a repeat that differs from a high-coverage (>M) copy of this repeat by a substitution of a block of t consecutive nucleotides. Without knowing that one deals with a repeat, the orphan elimination procedure would first detect two orphans, one of them ending in the first position of the block, and the other one starting in the last position of the block. If the orphans are eliminated without checking the “at most A corrections per read” condition, these two error corrections will shrink the block to the size (t−2) and will create two new orphans in the beginning and the end of this shrunk block. At the next step, this procedure would correct the first and the last nucleotides in the shrunk block, and, in just t/2 steps, erase the differences between two copies of the repeat.

Of course, for long bacterial genomes many “bad” events that may look improbable happen and there are two types of errors that are prone to orphan elimination. They require a few coordinated error corrections since single error corrections do not lead to a significant reduction in the size of Sₗ and thus may be missed by the greedy orphan elimination. These errors comprise: (i) consecutive or closely-spaced errors in the same read; and (ii) the same error with high multiplicity (>M) at the same genome position in different reads.

The first type of error is best addressed by solving the Spectral Alignment Problem to identify reads that require less than Δ error corrections. Some reads from the N. meningitidis project have very poor spectral alignment. These reads are likely to represent contamination, vector, isolated reads, or an error in the sequencing pipeline. All these reads are of limited interest and should be discarded. In fact, it is a common practice in sequencing centers to discard such “poor-quality” reads and this approach is adopted in this invention. Although deleting poor-quality reads may slightly reduce the amount of available sequencing information, it greatly simplifies the assembly process. Another important advantage of spectral alignment is an ability to identify the chimerical reads. Such reads are characterized by good spectral alignments of the prefix and suffix parts that, however, cannot be extended to a good spectral alignment of the entire read. EULER breaks the chimerical reads into two or more pieces and preserves the sequencing information from their prefix and suffix parts.

The second type of error reflects the situation with M identical errors in different reads corresponding to the same genome position and generating an erroneous l-tuple with high multiplicity. For example, if both the correct and erroneous l-tuples have multiplicity 3 (with default threshold M=2), it is hard to decide whether one deals with a unique region (with coverage 6) or with two copies of an imperfect repeat (each with coverage 3). In the N. meningitidis project there were 1,610 errors with multiplicity 3 and larger.

Eulerian Superpath Problem

As described above, the idea of the Eulerian path approach to SBH is to construct a graph whose edges correspond to l-tuples and to find a path visiting every edge of this graph exactly once.

Given a set of reads S={S₁, . . . , Sₙ}, define the de Bruijn graph G(Sₗ) with vertex set S₍ₗ₋₁₎ (the set of all (l−1)-tuples from S) as follows. An (l−1)-tuple v∈S₍ₗ₋₁₎ is joined by a directed edge with an (l−1)-tuple w∈S₍ₗ₋₁₎, if Sₗ contains an l-tuple for which the first l−1 nucleotides coincide with v and the last l−1 nucleotides coincide with w. Each l-tuple from Sₗ corresponds to an edge in G.

If S contains the only sequence s₁, then this sequence corresponds to a path visiting each edge of G exactly once, an Eulerian path.

Finding Eulerian paths is a well-known problem that can be efficiently solved. The reduction from SBH to the Eulerian path problem described above assumes unit multiplicities of edges (no repeating l-tuples) in the de Bruijn graph (see below for a discussion on multiple edges). It is assumed herein that S contains a direct complement of every read.

In such case, G(Sₗ) includes reverse complement for every l-tuple and the de Bruijn graph can be partitioned into 2 subgraphs, one corresponding to a “canonical” sequence, and another one to its reverse complement.

With real data, the errors hide the correct path among many erroneous edges. The overall number of vertices in the graph corresponding to the error-free data from the NM project is 4,039,248 (roughly twice the length of the genome), while the overall number of vertices in the graph corresponding to real sequencing reads is 9,474,411 (for 20-mers). After the error-correction procedure this number is reduced to 4,081,857.

A vertex v is called a source if indegree (v)=0, a sink if outdegree (v)=0. A vertex v is called an intermediate vertex if indegree (v)=outdegree (v)=1 and a branching vertex if indegree (v)•outdegree (v)>1. A vertex is called a knot if indegree (v)>1 and outdegree (v)>1. For the N. meningitidis genome, the de Bruijn graph has 502,843 branching vertices for original reads (for l-tuple size 20). Error correction simplifies this graph and leads to a graph with 382 sources and sinks and 12,175 branching vertices. The error-free reads lead to a graph with 11,173 branching vertices.

Since the de Bruijn graph gets very complicated even in the error-free case, taking into account the information about which l-tuples belong to the same reads (that was lost after the construction of the de Bruijn graph) helps to untangle this graph.

A repeat vₗ, . . . vₙ and a system of paths overlapping with this repeat. The uppermost path contains the repeat and defines the correct pairing between the corresponding entrance and exit. If this path were not present, the repeat vₗ . . . vₙ would become a tangle

A path v₁ . . . vₙ in the de Bruijn graph is called a repeat if indegree (v₁)>1, outdegree (vₙ)>1, and outdegree (vᵢ)=1 for 1≦i≦n−1 (shown on FIG. 2). Edges entering the vertex vᵢ are called entrances into a repeat while edges leaving the vertex vₙ are called exits from a repeat. An Eulerian path visits a repeat a few times and every such visit defines a pairing between an entrance and an exit. Repeats may create problems in fragment assembly since there are a few entrances in a repeat and a few exits from a repeat but it is not clear which exit is visited after which entrance in the Eulerian path. However, most repeats can be resolved by read-paths (i.e., paths in the de Bruijn graph that correspond to sequencing reads) covering these repeats.

A read-path covers a repeat if it contains an entrance into this repeat and an exit from this repeat. Every covering read-path reveals some information about the correct pairings between entrances and exits. However, some parts of the de Bruijn graph are impossible to untangle due to long perfect repeats that are not covered by any read-paths. A repeat is called a tangle if there is no read-path containing this repeat (FIG. 2).

Tangles create problems in fragment assembly since pairings of entrances and exits in a tangle cannot be resolved via the analysis of read-paths. To address this issue a generalization of the Eulerian Path Problem is formulated as follows.

Eulerian Superpath Problem.

Given an Eulerian graph and a collection of paths in this graph, find an Eulerian path in this graph that contains all these paths as subpaths. The classical Eulerian Path Problem is a particular case of the Eulerian Superpath Problem with every path being a single edge. To solve the Eulerian Superpath Problem both the graph G and the system of paths P in this graph are transformed into a new graph Gₗ with a new system of paths P₁. Such transformation is called equivalent if there exists a one-to-one correspondence between Eulerian superpaths in (G, P) and (G₁, Pₗ). The goal is to make a series of equivalent transformations

(G, P)→(G₁, Pₗ)→. . . →(Gₖ,Pₖ)

that lead to a system of paths Pₖ with every path being a single edge. Since all transformations on the way from (G, P) to (Gₖ,Pₖ) are equivalent, every solution of the Eulerian Path Problem in (Gₖ,Pₖ) provides a solution of the Eulerian Superpath Problem in (G, P).

Described below is a simple equivalent transformation that solves the Eulerian Superpath Problem in the case when the graph G has no multiple edges.

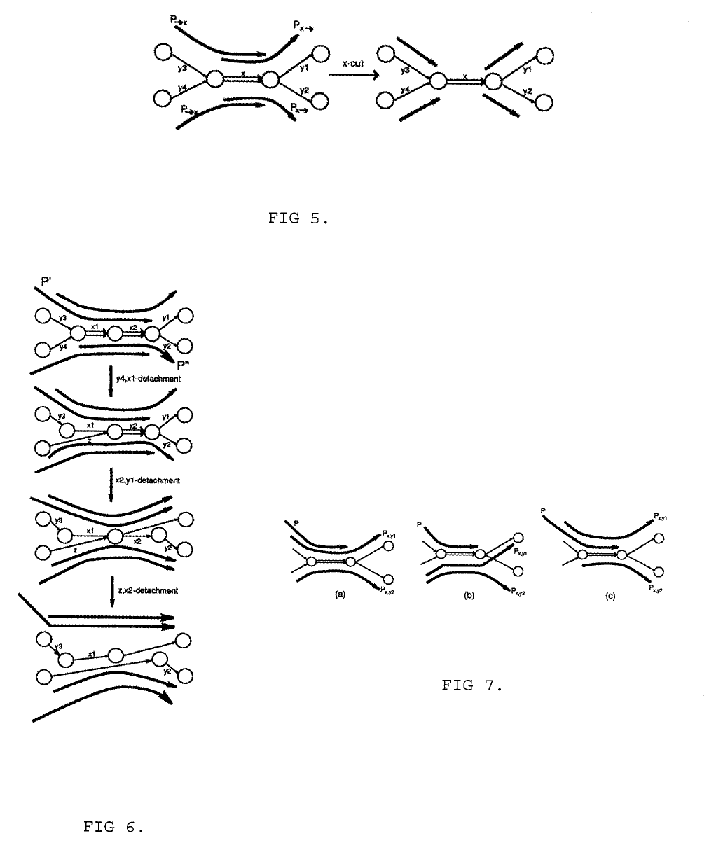

Let x=(vᵢₙ, v_{mid}) and y=(v_{mid}, vₒᵤₜ) be two consecutive edges in graph G and let P_{x,y} be a collection of all paths from P that include both these edges as a subpath. Define P_{→x} as a collection of paths from P that end with x and P_{y→} as a collection of paths from P that start with y. The x,y-detachment is a transformation that adds a new edge z=(vᵢₙ, vₒᵤₜ) and deletes the edges x and y from G, as shown on FIG. 3.

This detachment alters the system of paths P as follows: (i) substitute z instead of x, y in all paths from P_{x,y}, (ii) substitute z instead of x in all paths from P_{→x}, and (iii) substitute z instead of y in all paths from P_{y→}. Informally, detachment bypasses the edges x and y via a new edge z and directs all paths in P_{→x}, P_{y} and P_{x,y} through z.

Since every detachment reduces the number of edges in G, the detachments will eventually shorten all paths from P to single edges and will reduce the Eulerian Superpath Problem to the Eulerian Path Problem.

However, in the case of graphs with multiple edges, the detachment procedure described above may lead to non-equivalent transformations. In this case, the edge x may be visited many times in the Eulerian path and it may or may not be followed by the edge y on some of these visits. That's why, in case of multiple edges, “directing” all paths from the set P_{→x} through a new edge z may not be an equivalent transformation. However, if the vertex v_{mid} has no other incoming edges but x, and no other outgoing edges but y, then x,y-detachment is an equivalent transformation even if x and y are multiple edges. In particular, detachments can be used to reduce every repeat to a single edge.

It is important to realize that even in the case when the graph G has no multiple edges, the detachments may create multiple edges in the graphs G₁, . . . , Gₖ (for example, if the edge (vᵢₙ, vₒᵤₜ) were present in the graph prior to the detachment procedure). However, such multiple edges do not pose problems, since in this case it is clear what instance of the multiple edge is used in every path, as discussed below.

As shown on FIG. 4, in the case when x is a multiple edge, x,y1-detachment is an equivalent transformation if P_{→x} is empty. If P_{→x} is not empty, it is not clear whether the last edge of a path P∈P_{→x} should be assigned to z or to x.

For illustration purposes, an example is a simple case when the vertex v_{mid} has the only incoming edge x=(vᵢₙ, v_{mid}) with multiplicity 2 and two outgoing edges y1=(v_{mid}, vₒᵤₜ₁) and y2=(v_{mid}, vₒᵤₜ₂), each with multiplicity 1, as shown on FIG. 4. In this case, the Eulerian path visits the edge x twice, in one case it is followed by y1 and in another case it is followed by y2. Consider an x,y1-detachment that adds a new edge z=(vᵢₙ, vₒᵤₜ₁) after deleting the edge y1 and one of two copies of the edge x. This detachment (i) shortens all paths in P_{x,y1} by substitution of x,y1 by a single edge z and (ii) substitutes z instead of y1 in every path from P_{y1→}. This detachment is an equivalent transformation if the set P_{→x} is empty. However, if P_{→x} is not empty, it is not clear whether the last edge of a path P∈P_{→x} should be assigned to the edge z or to the remaining copy of edge x.

To resolve this dilemma, one has to analyze every path P∈P_{→x} and to decide whether it “relates” to P_{x,y1} (in this case it should be directed through z) or to P_{x,y2} (in which case it should be directed through x). “Relates” to P_{x,y1}(P_{x,y2}) means that every Eulerian superpath visits y1 (y2) right after visiting P.

To introduce further definitions, two paths are called consistent if their union is a path again (there are no branching vertices in their union). A path P is consistent with a set of paths P if it is consistent with all paths in P and inconsistent otherwise (i.e. if it is inconsistent with at least one path in P). There are three possibilities as shown on FIG. 5: (a) P is consistent with exactly one of the sets P_{x,y1} and P_{x,y2}; (b) P is inconsistent with both P_{x,y1} and P_{x,y2}; and (c) P is consistent with both P_{x,y1} and P_{x,y2}.

As shown on FIG. 5(a), P is consistent with P_{x,y1}, but inconsistent with P_{x,y2}; as shown on FIG. 5(b), P is inconsistent with both P_{x,y1} and P_{x,y2}; and as shown on FIG. 5(c), P is consistent with both P_{x,y1} and P_{x,y2}.

In the first case, the path P is called resolvable since it can be unambiguously related to either P_{x,y1} or P_{x,y2}. If P is consistent with P_{x,y1} and inconsistent with P_{x,y2}, then P should be assigned to the edge z after x,y1-detachment (substitute x by z in P). If P is inconsistent with P_{x,y1} and consistent with P_{x,y2}, then P should be assigned to the edge x (no action taken). An edge x is called resolvable if all paths in P_{→x} are resolvable. If the edge x is resolvable then the described x,y-detachment is an equivalent transformation after the correct assignments of last edges in every path from P_{→x}. In the analysis of the NM project, it was found that 18,026 among 18,962 edges in the de Bruijn graph are resolvable. Although the notion of resolvable path was defined hereinabove for a simple case (see FIG. 3) (i.e., when the edge x has multiplicity 2), it can be generalized for edges with arbitrary multiplicities. The second condition (P is inconsistent with both (P_{x,y1} and P_{x,y2}) implies that the Eulerian Superpath Problem has no solution, i.e., sequencing data are inconsistent. Informally, in this case P, P_{x,y1} and P_{x,y2} impose three different scenarios for just two visits of the edge x. After discarding the poor-quality and chimerical reads, there was no encountering this condition in the analysis of the NM project.

The last condition (P is consistent with both P_{x,y1} and P_{x,y2}) corresponds to the most difficult situation and deserves a special discussion. If this condition holds for at least one path in P_{→x} the edge x is called unresolvable and the analysis of this edge is postponed until all resolvable edges are analyzed. It may turn out that equivalent transformation of other resolvable edges will make the edge x resolvable. FIG. 6 illustrates that equivalent transformations may resolve previously unresolvable edges.

As demonstrated on FIG. 6, in the graph on the top, the edge x2 is not resolvable (P′∈P_{→x2} is consistent with both P_{x2,y1} and P_{x2,y2}) and the edge x1 is resolvable (P″∈P_{x1→} is consistent with P_{y4,x1} and inconsistent with P_{y3,x1}). Therefore y4,x1-detachment is an equivalent transformation substituting y4 and x1 by z (second graph on FIG. 6). This transformation makes x2 resolvable and opens a door for x2,y1-detachment (third graph on FIG. 6). Finally, z,x2-detachment leads to a simplification of the original graph.

However, some edges cannot be resolved even after the detachments of all resolvable edges are completed. Such situations usually correspond to tangles and they have to be addressed by another equivalent transformations called a cut. If x is a removable edge, then x-cut is an equivalent transformation shortening the paths in P, as shown on FIG. 7.

A fragment of graph G with 5 edges and four paths y3-x, y4-x, x-y1 and x-y2 is of interest, each path comprising two edges (shown on FIG. 7). In this case, P_{→x} comprises two paths, y3-x and y4-x, and each of these paths is consistent with both P_{x,y1} and P_{x,y2}. In fact, in this symmetric situation, x is a tangle and there is no information available to relate any of path y3-x and y4-x to any of paths x-y1 and x-y2. Therefore, it may happen that no detachment is an equivalent transformation in this case. To address this problem, another equivalent transformation is introduced that affects the system of paths P and does not affect the graph G itself.

An edge x=(v,w) is removable if (i) it is the only outgoing edge for v and the only incoming edge for w and (ii) x is either initial or terminal edge for every path P∈P containing x. An x-cut transforms P into a new system of paths by simply removing x from all paths in P_{→x} and P_{x→}.

In the case illustrated on FIG. 7, x-cut shortens the paths x-y1, x-y2, y3-x, and y4-x to single-edge paths y1, y2, y3, and y4. It is easy to see that an x-cut of a removable edge is an equivalent transformation, i.e., every Eulerian superpath in (G,P) corresponds to an Eulerian superpath in (G,Pₗ) and vice versa. Cuts proved to be a powerful technique to analyze tangles that are not amenable to detachments. Detachments reduce such tangles to single unresolvable edges that turned out to be removable in the analysis of bacterial genomes. It allows to reduce the Eulerian Superpath Problem to the Eulerian Path Problem for all studied bacterial genomes.

It is particularly emphasized that the method of this invention of equivalent graph transformations for fragment assembly is very different from the graph reduction techniques for fragment assembly suggested in prior art.

### Examples

The method of this invention was tested with real sequencing data from the C. jejuni (CJ), N. meningitidis (NM), and L. lactis (LL) genomes, all the genomes data described in the above-referenced prior art documents. The results are summarized in Table 1. These genomes were assembled in the prior art references either by Phrap (CJ and NM) or by gap4 (LL) software and required substantial finishing efforts to complete the assembly and to correct the assembly errors.

TABLE 1
Assembly of reads from (C. jejuni) (CJ), (N. meningitidis)
(NM), and (L. lactis) (LL) sequencing projects.

| Project | CJ | NM | LL |
|---|---|---|---|
| Genome length | 1,641,481 | 2,184,406 | 2,365,590 |
| Average read length | 502 | 400 | 568 |
| No. of islands in reads coverage | 24 | 79 | 6 |
| Coverage | 10.3 | 9.7 | 6.4 |
| No. of reads | 33708 | 53263 | 26532 |
| No. of poor quality reads | 431 | 251 | 128 |
| No. of chimeric reads | 611 | 874 | 935 |
| Error rate | 1.6% | 1.2% | 2.1% |
| No. of errors per read (average) | 8.0 | 4.8 | 11.9 |
| No. of reads after: | | | |
| orphan elimination/spectral align. | 0.84 | 0.36 | 1.4 |
| correcting high-multiplicity errors | 0.41 | 0.30 | 0.78 |
| filtering poor-quality and | 0.17 | 0.11 | 0.32 |
| chimeric reads | | | |
| After error correction: | | | |
| Coverage | 10.1 | 9.0 | 6.3 |
| No. of reads | 32666 | 52138 | 25469 |
| No. consistent errors per read | 0.16 | 0.10 | 0.27 |
| No. inconsistent errors per read | 0.01 | 0.01 | 0.05 |
| % of corrected errors | 97.9% | 97.7% | 97.3 |
| Before solving the | | | |
| Eulerian Superpath Problem: | | | |
| No. of vertices in de Bruijn graph | 3,197,687 | 4,081,857 | 4,430,803 |
| (1 = 20) | | | |
| No. of branching vertices (1 = 20) | 2132 | 12175 | 4873 |
| After solving the | | | |
| Eulerian Superpath Problem: | | | |
| No. of vertices in de Bruijn graph | 126 | 999 | 148 |
| (1 = 20) | | | |
| No. of branching vertices (1 = 20) | 30 | 617 | 124 |
| No. of sources/sinks (1 = 20) | 96 | 382 | 24 |
| No. of edges | 74 | 1028 | 63 |
| No. of connected single-edge | 26 | 112 | 48 |
| components (1 = 20) | | | |
| No. of connected components | 33 | 122 | 62 |
| (1 = 20) | | | |
| No. of tangles: | 2 | 31 | 16 |
| Overall multiplicity of tangles | 5 | 126 | 61 |
| Maximum multiplicity of tangles | 3 | 21 | 9 |
| Running time (hours) | 3 | 5 | 6 |
| 8 CPU 9GB Sun Enterprise | | | |
| E4500/E5500 | | | |

Orphan elimination and spectral alignment already provide a tenfold reduction in the error rate. However, further reductions in error rate are important since they simplify the de Bruijn graph and lead to the efficient solution of the Eulerian Superpath Problem. After the error correction is completed, the number of errors is reduced by a factor of 35-50 making reads almost error-free. To check the accuracy of the assembled contigs each assembled contig is fit into the genomic sequence via local sequence alignment as known by those skilled in the art. A contig is assumed to be correct if it fits into a genomic sequence as a continuous fragment with a small number of errors. Inability to fit a contig into the genomic sequence with a small number of errors indicates that the contig is misassembled.

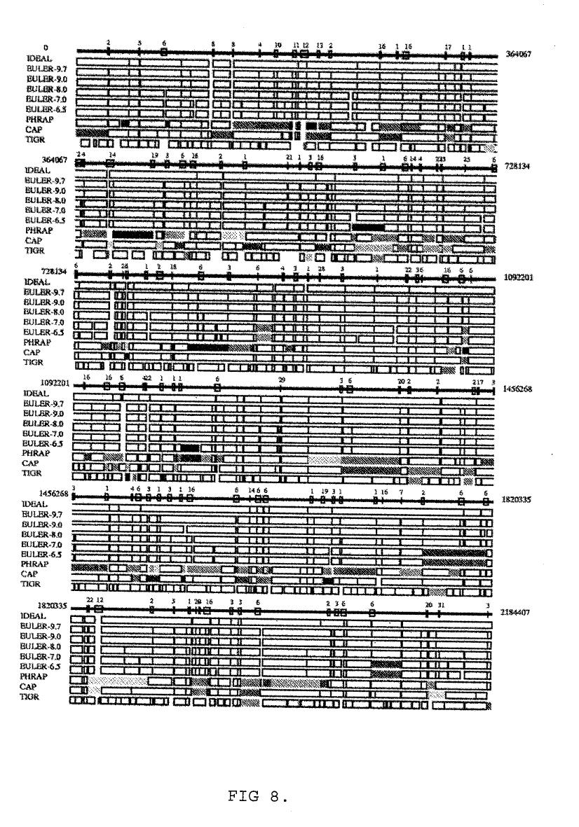

For example, Phrap misassembles 17 contigs in the N. meningitidis sequencing project, each contig containing up to four fragments from different parts of the genome. The misassembled contig is broken into two or more fragments and fit each fragment separately into different locations in the genome. To compare EULER with other fragment assembly algorithms, Phrap, CAP3 and TIGR assemblers are used(default parameters) for CJ, NM, and LL sequencing projects, as shown in Table 2 and FIG. 8.

TABLE 2
Comparison of different software tools for fragment assembly. IDEAL is
an imaginary assembler that outputs the collection of islands in clone coverage as contigs.
(In the IDEAL column the second number in the pair shows the overall multiplicity of tangles).

| | | IDEAL | EULER | Phrap | CAP3 | TIGR |
|---|---|---|---|---|---|---|
| CJ | No. contigs/No. misassembled contigs | 24/5 | 29/0 | 33/2 | 54/3 | >300/>10 |
| | Coverage by contigs (%) | 99.5 | 96.7 | 94.0 | 92.4 | 90.0 |
| | Coverage by misassebled contigs (%) | — | 0.0 | 16.1 | 13.6 | 1.2 |
| NM | No. contigs/No. misassembled contigs | 79/126 | 149/0 | 160/17 | 163/14 | >300/9 |
| | Coverage by contigs (%) | 99.8 | 99.1 | 98.6 | 97.2 | 87.4 |
| | Coverage by misassebled contigs (%) | — | 0.0 | 10.5 | 9.2 | 1.3 |
| LL | No. contigs/No. misassembled contigs | 6/61 | 58/0 | 62/10 | 85/8 | 245/2 |
| | Coverage by contigs (%) | 99.9 | 99.5 | 97.6 | 97.0 | 90.4 |
| | Coverage by misassebled contigs (%) | — | 0.0 | 19.0 | 11.4 | 0.7 |

Every box in FIG. 8 corresponds to a contig in the NM assembly produced by these programs. Boxes in the IDEAL assembly correspond to islands in the read coverage. Boxes of the same shade show misassembled contigs, for example two identically shaded boxes in different places show the positions of contigs that were incorrectly assembled into a single contig. In some cases, a single shaded box shows a contig that was assembled incorrectly (i.e., there was a rearrangement inside this contig). The tangles are indicated by numbered boxes at the solid line showing the genome. For example, in the CJ sequencing project, there are 2 tangles, one with multiplicity 3 and another with multiplicity 2. EULER produces 29 contigs and it can be proved that it is an optimal assembly, i.e., no program can produce an assembly with a smaller number of contigs. The CJ sequencing project has 24 islands in the coverage and the overall multiplicity of tangles in this project is 5. Since all these tangles belong to different islands, the lower bound for the number of contigs in any valid assembly is 24+5=29. An important advantage of EULER is that it suggests five PCR experiments that resolve all tangles and generate the IDEAL assembly. This feature is particularly important for the LL project since approximately 50 PCR experiments would reduce the number of reported contigs by a factor of tenfold.

The C. jejuni sequencing project is relatively simple due to an unusually low number of long perfect repeats. However, even in this relatively simple case, Phrap, CAP3, and TIGR made assembly errors. This genome contains only four long repeated sequences with similarity 90 and higher. However, only two of these four repeats contain long perfect sub-repeats (tangles) and cannot be resolved even theoretically. EULER resolves two other repeats and breaks the contigs at the positions of tangles to avoid potential assembly errors. N. meningitidis contains hundreds of repeats, some of them very long. Among these repeats 31 are tangles (each repeating four times on average) and extensive finishing work was required to untangle them.

L. lactis has a fewer number of repeats than N. meningitidis but it poses a different challenge: low coverage and high error rate. Although EULER successfully assembled this genome, further reduction in coverage and increase in error rate would “break” EULER. Table 3 and FIG. 8 study the question: “How low can EULER go in coverage?” by deleting some reads and running EULER on this reduced set of reads. These results indicate that EULER produces a better assembly with coverage 7-8 than other programs with the full coverage 9.7. The analysis of errors made by EULER in these simulated low coverage projects indicates that they are “reporting” rather than algorithmic errors. The problem is that in the low-coverage regions (e.g., regions with coverage 1), it is theoretically impossible to filter out the chimerical reads (some chimerical reads may look like perfectly legitimate reads). If such low-coverage regions are not reported by EULER (they are subject to finishing efforts anyway), then it becomes error-free again.

TABLE 3
EULER's performance with reduced coverage (NM sequencing project).

| | | | | | |
|---|---|---|---|---|---|
| No. of reads | 53263 | 49420 | 43928 | 38437 | 35690 |
| Coverage | 9.7 | 9.0 | 8.0 | 7.0 | 6.5 |
| No. contigs/No. misassembled contigs | 149/0 | 152/0 | 175/0 | 182/2 | 185/3 |
| Coverage by contigs (%) | 99.5 | 99.1 | 98.5 | 95.5 | 94.8 |
| Coverage by misassembled contigs (%) | 0.0 | 0.0 | 0.0 | 4.1 | 6.2 |

Conclusion

Finishing is a bottleneck in large-scale DNA sequencing. Of course, finishing is an unavoidable step to extend the islands and to close the gaps in read coverage. However, the existing programs produce much more contigs then the number of islands thus making finishing more complicated than necessary. What is worse, these contigs are often assembled incorrectly thus leading to time-consuming contig verification step. EULER bypasses this problem since the Eulerian Superpath approach transforms imperfect repeats into different paths in the de Bruijn graph. As a result, EULER does not even notice repeats unless they are long perfect repeats, i.e., when the corresponding paths cannot be separated. Tangles are theoretically impossible to resolve and therefore some additional biochemical experiments are needed to correctly position them.

Difficulties in resolving repeats led to the introduction of the double-barreled DNA sequencing and the breakthrough genome sequencing efforts at Celera known to those skilled in the art. The Celera assembler is a two-stage procedure that includes masking repeats at the overlap-layout-consensus stage with further ordering of contigs via the double-barreled information. EULER has excellent scaling potential and the work on integrating EULER with the double-barreled data is now in progress. In fact, the complexity of EULER is mainly defined by the number of tangles rather than the number of repeats/length of the genome. It is believed that assembly of some simple eukaryotic genomes with a small number of tangles may be even less challenging for EULER than the assembly of the N. meningitidis genome. EULER does not require masking the repeats but instead provides a clear view of the structure of the perfect repeats (tangles) in the genome. These tangles may be resolved either by double-barreled information or by additional PCR experiments. These PCR experiments do not even require sequencing of PCR products but can be done through simple length measurements of PCR products. Since only a few PCR primers are needed to resolve each tangle, this “on-demand” finishing method potentially may reduce the cost of DNA sequencing as compared to the double-barreled approach.

Since reliable programs for pure shotgun assembly of complete genomes are still unavailable, the biologists are forced to do time-consuming mapping, verification, and finishing experiments to complete the assembly. As a result, most bacterial sequencing projects today start from mapping, a rather time-consuming step. Of course, mapping provides some insurance against assembly errors but it is not a 100%-proof insurance and it does not come for free. The only computational reason for using mapping information is to correct assembly errors and to resolve some repeats. EULER promises to make mapping unnecessary for sequencing applications, since it does not make errors, resolve all repeats but tangles, and suggests very few PCR experiments to resolve tangles. The amount of experimental efforts associated with these “on demand” experiments is much smaller than with mapping efforts.

Having described the invention in connection with several embodiments thereof, modification will now suggest itself to those skilled in the art. As such, the invention is not to be limited to the described embodiments except as required by the appended claims.

We claim:

1. A method for correcting errors in original sequencing reads of a DNA, said method comprising:
performing an error detection and correction, wherein said error correction is conducted prior to assembling such reads in a contiguous piece.

2. A method of assembling a puzzle from original pieces thereof, the method comprising:
altering said original pieces.

3. The method as claimed in claim 2, wherein said puzzle comprises a DNA sequence.

4. The method as claimed in claim 2 or 3, wherein said altering said original pieces comprises dismembering said original pieces into sub-pieces, each sub-piece being smaller in size than said original piece from which said sub-piece originated.

5. The method as claimed in claim 3, wherein said original pieces comprise reads of said DNA sequence.
